(19) United States
(12) Patent Application Publication
(10) Pub. No.: US 2025/0222182 A1
(43) Pub. Date: Jul. 10, 2025

(54) Simulative Breastfeeding Device

(71) Applicant: Justin Moreno, Chesterton, IN (US)

(72) Inventor: Justin Moreno, Chesterton, IN (US)

(21) Appl. No.: 18/953,368

(22) Filed: Nov. 20, 2024

Related U.S. Application Data
(60) Provisional application No. 63/618,585, filed on Jan. 8, 2024.

(51) Int. Cl. A61M 1/06; A41C 3/00; A41C 3/04

(52) U.S. Cl. CPC A61M 1/068; A41C 3/0035; A41C 3/04

(57) ABSTRACT

A simulative breastfeeding device is provided. The device is comprised of an upper body-worn garment. The garment has an interior pocket that receives at least one reservoir. The reservoir is shaped like a breast and is comprised of an areola and a nipple. During use, the reservoir can be filled with breast milk and/or infant formula and placed into the interior pocket such that the areola and nipple extend outwards from an opening in the garment. Therefore, a user can wear the garment and breastfeed an infant (in a simulated manner) via the reservoir and nipple.

### CROSS-REFERENCE TO RELATED APPLICATION

The present application claims priority to, and the benefit of, U.S. Provisional Application No. 63/618,585, which was filed on Jan. 8, 2024, and is incorporated herein by reference in its entirety.

### FIELD OF THE INVENTION

The present invention relates generally to the field of breastfeeding devices. More specifically, the present invention relates to a simulative breastfeeding device that provides an upper body-worn garment that receives a reservoir with a nipple that can deliver breastmilk and/or formula to an infant. Accordingly, the present disclosure makes specific reference thereto. Nonetheless, it is to be appreciated that aspects of the present invention are also equally applicable to other like applications, devices, and methods of manufacture.

### BACKGROUND

Caring for infants often involves addressing the challenge of feeding, particularly when many babies display a strong aversion to using a feeding bottle. This situation can be quite stressful for caretakers, especially considering that breastfeeding, widely recognized as the optimal method of infant nutrition, is not a viable option for everyone. The reasons for this vary widely and include a range of personal, medical, and social factors.

For some mothers, physical health complications can make breastfeeding difficult or impossible. Conditions such as insufficient glandular tissue, previous breast surgeries, or infections like mastitis can significantly hinder the ability to nurse. Additionally, certain medical treatments and medications may be incompatible with breastfeeding, necessitating alternative feeding methods for the infant.

Work commitments and lifestyle factors also play a crucial role. In today's fast-paced society, where maternity leave is often limited, many mothers are forced to return to work shortly after childbirth, making regular breastfeeding sessions challenging. Pumping breast milk is a solution for some, but it requires access to appropriate facilities at work and can be time consuming and physically uncomfortable.

Moreover, the reality of modern family structures means that primary caregivers are not always the biological mothers. Adoptive parents, fathers, grandparents, or other family members might take on the role of the primary caretaker. In these cases, bottle feeding becomes an essential method of nourishing the infant.

Beyond these considerations, there is the psychological impact on both the baby and the caregiver. For infants, the transition from breast to bottle can be a source of distress, leading to feeding aversions and nutritional concerns. For caregivers, the pressure to adhere to societal expectations of breastfeeding, coupled with the challenges of bottle refusal by the infant, can lead to feelings of inadequacy and stress.

Therefore, there exists a long-felt need in the art for a breastfeeding device. There also exists a long-felt need in the art for a simulative breastfeeding device. In addition, there exists a long-felt need in the art for a simulative breastfeeding device that allows a user without breasts (or with the inability to produce breast milk) to breastfeed. More specifically, there exists a long-felt need in the art for a simulative breastfeeding device that allows a user without breasts (or with the inability to produce breast milk) to breastfeed via a simulative breast.

The subject matter disclosed and claimed herein, in one embodiment thereof, comprises a simulative breastfeeding device. The device is comprised of an upper body-worn garment. The garment has an interior pocket that receives at least one reservoir. The reservoir is shaped like a breast and is comprised of an areola and a nipple. During use, the reservoir can be filled with breast milk and/or infant formula and placed into the interior pocket such that the areola and nipple extend outwards from an opening in the garment. Therefore, a user can wear the garment and breastfeed an infant (in a simulated manner) via the reservoir and nipple.

In this manner, the simulative breastfeeding device of the present invention accomplishes all the foregoing objectives and provides a breastfeeding device. More specifically, the device allows a user without breasts (or with the inability to produce breast milk) to breastfeed. More specifically, the device does so via a simulated breast.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a simulative breastfeeding device comprised of an upper body-worn garment that receives at least one breast-shaped reservoir in an interior pocket. The reservoir can be filled with infant bottle formula (and/or breast milk) to allow an infant to feed from the reservoir via at least one nipple.

The garment generally resembles a sports bra and is comprised of at least one shoulder strap. The garment may further be colored in a skin tone shade. The garment may be adjustable via at least one rear fastener and at least one front fastener.

The device is also comprised of at least one reservoir that is preferably shaped like a breast. During use, the reservoir is received by an interior pocket of the garment wherein at least one nipple of the reservoir extends through the garment via at least one opening. The reservoir can be filled with infant feeding formula and/or breast milk via at least one opening. To prevent colic while feeding, the reservoir may be comprised of at least one vent that prevents an infant from swallowing air while feeding from the reservoir. The reservoir is further comprised of at least one areola with at least one nipple. The nipple is comprised of at least one opening that allows an infant to suck formula out of the reservoir via the nipple.

The present invention is also comprised of a method of using the device. First, a device is provided comprised of a garment comprised of at least one interior pocket that receives at least one reservoir comprised of at least one nipple comprised of at least one opening. Then, the reservoir can be filled with an infant feeding formula and/or breastmilk via an opening. Then, a vent of the reservoir can be opened to prevent colic and the reservoir can be placed into an interior pocket of the garment such that the nipple and areola protrude through an opening in the garment. Then, a user can wear the garment around their torso/upper body and adjust/secure the garment via at least one fastener. Finally, a user can breastfeed an infant by allowing the infant to suck on the nipple to release formula/breastmilk out of the reservoir.

Accordingly, the simulative breastfeeding device of the present invention is particularly advantageous as it provides a breastfeeding device. More specifically, the device allows a user without breasts (or with the inability to produce breast milk) to breastfeed. More specifically, the device does so via a simulated breast. In this manner, the simulative breastfeeding device provides a solution to allow all users to breastfeed an infant.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a simulative breastfeeding device of the present invention in accordance with the disclosed architecture;

FIG. 2 illustrates a perspective view of a garment of one potential embodiment of a simulative breastfeeding device of the present invention while open in accordance with the disclosed architecture;

FIG. 3 illustrates a rear view of a garment of one potential embodiment of a simulative breastfeeding device of the present invention while worn by a user in accordance with the disclosed architecture;

FIG. 4 illustrates a side perspective view of a reservoir of one potential embodiment of a simulative breastfeeding device of the present invention in accordance with the disclosed architecture;

FIG. 5 illustrates a rear perspective view of a reservoir of one potential embodiment of a simulative breastfeeding device of the present invention in accordance with the disclosed architecture; and

FIG. 6 illustrates a flowchart of a method of using one potential embodiment of a simulative breastfeeding device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a breastfeeding device. There also exists a long-felt need in the art for a simulative breastfeeding device. In addition, there exists a long-felt need in the art for a simulative breastfeeding device that allows a user without breasts (or with the inability to produce breast milk) to breastfeed. More specifically, there exists a long-felt need in the art for a simulative breastfeeding device that allows a user without breasts (or with the inability to produce breast milk) to breastfeed via a simulative breast.

The present invention, in one exemplary embodiment, is comprised of a simulative breastfeeding device. The device is comprised of an upper body-worn garment that receives at least one breast-shaped reservoir in an interior pocket. The reservoir can be filled with infant bottle formula (and/or breast milk) to allow an infant to feed from the reservoir via at least one nipple.

The garment generally resembles a sports bra and is comprised of at least one shoulder strap. The garment may further be colored in a skin tone shade and may be adjustable via at least one rear fastener and at least one front fastener.

The device is also comprised of at least one reservoir. The reservoir is preferably shaped like a breast. During use, the reservoir is received by an interior pocket of the garment wherein at least one nipple of the reservoir extends through the garment via at least one opening. The reservoir can be filled with infant feeding formula and/or breast milk via at least one opening. To prevent colic while feeding, the reservoir may be comprised of at least one vent that prevents an infant from swallowing air while feeding from the reservoir. The reservoir is further comprised of at least one areola with at least one nipple comprised of at least one opening that allows an infant to suck formula out of the reservoir via the nipple.

The present invention is also comprised of a method of using the device. First, a device is provided comprised of a garment comprised of at least one interior pocket that receives at least one reservoir comprised of at least one nipple comprised of at least one opening. Then, the reservoir can be filled with an infant feeding formula and/or breastmilk via an opening. Then, a vent of the reservoir can be opened to prevent colic and the reservoir can be placed into an interior pocket of the garment such that the nipple and areola protrude through an opening in the garment. Then, a user can wear the garment around their torso/upper body and adjust/secure the garment via at least one fastener. Finally, a user can breastfeed an infant by allowing the infant to suck on the nipple to release formula/breastmilk out of the reservoir.

Accordingly, the simulative breastfeeding device of the present invention is particularly advantageous as it provides a breastfeeding device. More specifically, the device allows a user without breasts (or with the inability to produce breast milk) to breastfeed. More specifically, the device does so via a simulated breast. In this manner, the simulative breastfeeding device provides a solution to allow all users to breastfeed an infant.

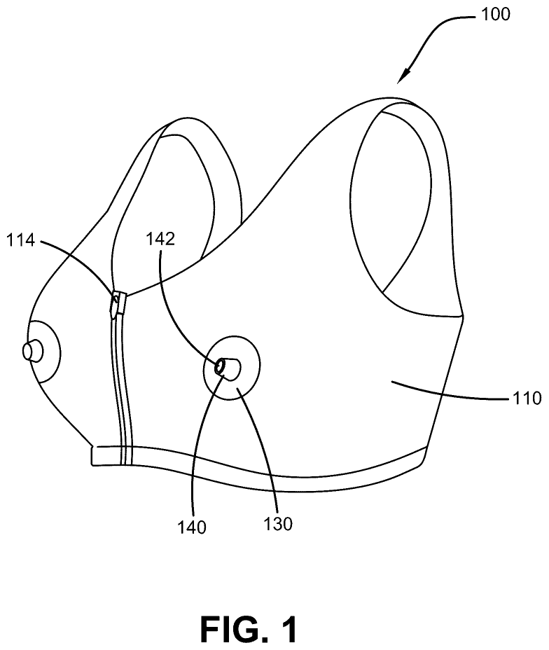

Referring initially to the drawings, FIG. 1 illustrates a perspective view of one potential embodiment of a simulative breastfeeding device 100 of the present invention in accordance with the disclosed architecture. The device 100 is comprised of an upper body-worn garment 110 that receives at least one breast-shaped reservoir 120 in an interior pocket 112. The reservoir 120 can be filled with infant bottle formula (and/or breast milk) to allow an infant to feed from the reservoir 120 via at least one nipple 140.

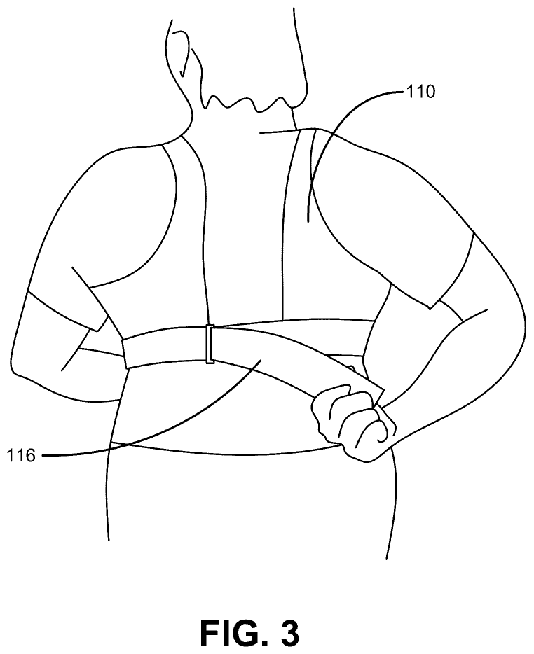

The garment 110 generally resembles a sports bra and is comprised of at least one shoulder strap 111. The strap 111 may be adjustable or non-adjustable. The garment 110 may be made from any material. The garment 110 may further be colored in a skin tone shade. The garment 110 may be adjustable via at least one rear fastener 116 such as, but not limited to, a strap, a buckle, a snap button fastener, a zipper, etc., as seen in FIG. 3. The garment 110 may further be adjustable/accessible via at least one front fastener 114, such as, but not limited to, a zipper.

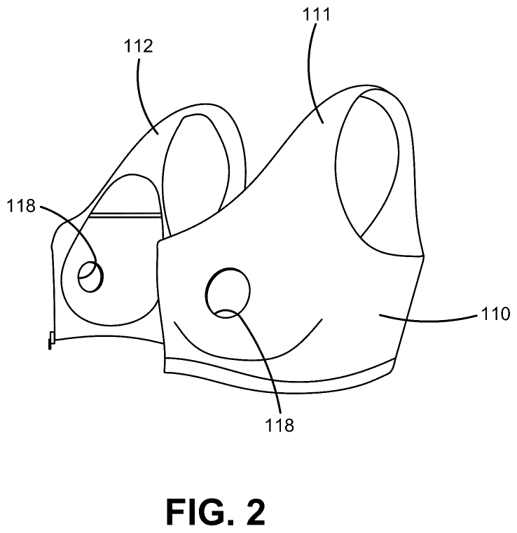

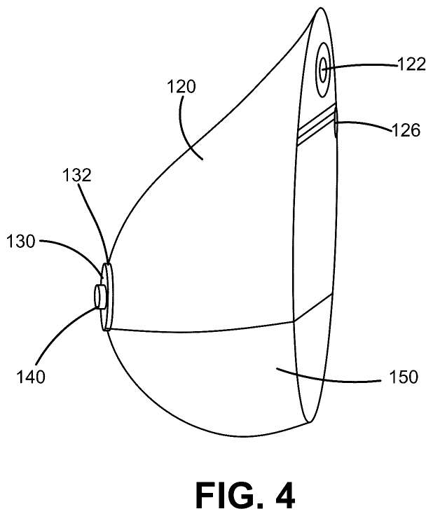

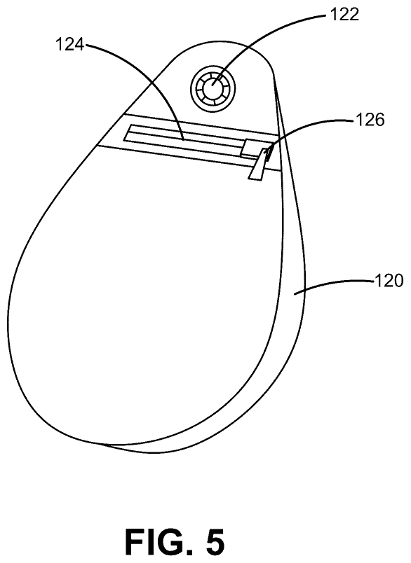

The device 100 is also comprised of at least one reservoir 120, as seen in FIG. 4 and FIG. 5. The reservoir 120 is preferably shaped like a breast. During use, the reservoir 120 is received by an interior pocket 112 of the garment 110 (as seen in FIG. 2), wherein at least one nipple 140 of the reservoir 120 extends through the garment 110 via at least one opening 118. The reservoir 120 may be made from any rigid, pliable, or semi-pliable material. In one embodiment, the reservoir 120 is comprised of at least one silicone area 150 located below and/or around the nipple 140 to simulate the feeling/sensation of a breast.

The reservoir 120 can be filled with infant feeding formula and/or breast milk via at least one opening 124. The opening 124 can be secured via a waterproof fastener 126 such as, but not limited to, a waterproof zipper, a waterproof cap, a waterproof seal, etc. To prevent colic while feeding, the reservoir 120 may be comprised of at least one vent 122 that prevents an infant from swallowing air while feeding from the reservoir 120.

The reservoir 120 is further comprised of at least one areola 130 with at least one nipple 140. The areola 130 is preferably comprised of a stepped perimeter edge 132. The edge 132 ensures the areola 130 is flush with the area of the garment 110 around the opening 118. The nipple 140 is comprised of at least one opening 142 that allows an infant to suck formula out of the reservoir 120 via the nipple 140. The nipple 140 and areola 130 may be made from any simulative texture such as but not limited to silicone.

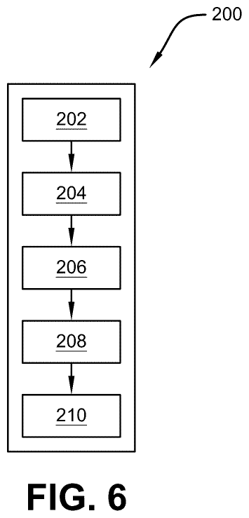

The present invention is also comprised of a method of using 200 the device 100, as seen in FIG. 6. First, a device 100 is provided comprised of a garment 110 comprised of at least one interior pocket 112 that receives at least one reservoir 120 comprised of at least one nipple 140 comprised of at least one opening 142 [Step 202]. Then, the reservoir 120 can be filled with an infant feeding formula and/or breastmilk via an opening 124 [Step 204]. Then, a vent 122 of the reservoir 120 can be opened to prevent colic and the reservoir 120 can be placed into an interior pocket 112 of the garment 110 such that the nipple 140 and areola 130 protrude through an opening 118 in the garment 110 [Step 206]. Then, a user can wear the garment 110 around their torso/upper body and adjust/secure the garment via at least one fastener 114,116 [Step 208]. Finally, a user can breastfeed an infant via allowing the infant to suck on the nipple 140 to release formula/breastmilk out of the reservoir 120 [Step 210].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “simulative breastfeeding device” and “device” are interchangeable and refer to the simulative breastfeeding device 100 of the present invention.

Notwithstanding the foregoing, the simulative breastfeeding device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the simulative breastfeeding device 100 as shown in the FIGS. are for illustrative purposes only, and that many other sizes and shapes of the simulative breastfeeding device 100 are well within the scope of the present disclosure. Although the dimensions of the simulative breastfeeding device 100 are important design parameters for user convenience, the simulative breastfeeding device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A simulative breastfeeding device comprising:
an upper body garment comprised of a first opening, a strap and an interior pocket;
a breast-shaped reservoir comprised of a second opening, an areola, and a nipple comprised of a third opening.

2. The simulative breastfeeding device of claim 1, wherein the breast-shaped reservoir is received by the interior pocket.

3. The simulative breastfeeding device of claim 1, wherein the upper body garment is comprised of a strap.

4. The simulative breastfeeding device of claim 1, wherein the upper body garment is comprised of a front fastener.

5. The simulative breastfeeding device of claim 1, wherein the upper body garment is comprised of a rear fastener.

6. The simulative breastfeeding device of claim 1, wherein the breast-shaped reservoir is comprised of a silicone area.

7. The simulative breastfeeding device of claim 1, wherein the areola is comprised of a stepped edge.

8. The simulative breastfeeding device of claim 1, wherein the upper body garment is skin colored.

9. A simulative breastfeeding device comprising:
an upper body garment comprised of a first opening, a strap, and an interior pocket;
a breast-shaped reservoir comprised of a second opening, a vent, an areola, and a nipple comprised of a third opening.

10. The simulative breastfeeding device of claim 9, wherein the breast-shaped reservoir is received by the interior pocket.

11. The simulative breastfeeding device of claim 9, wherein the upper body garment is comprised of an adjustable strap.

12. The simulative breastfeeding device of claim 9, wherein the upper body garment is comprised of a front fastener comprised of a zipper.

13. The simulative breastfeeding device of claim 9, wherein the upper body garment is comprised of a rear fastener comprised of a strap.

14. The simulative breastfeeding device of claim 9, wherein the breast-shaped reservoir is comprised of a silicone area.

15. The simulative breastfeeding device of claim 9, wherein the areola is comprised of a stepped edge.

16. The simulative breastfeeding device of claim 9, wherein the upper body garment is skin colored.

17. The simulative breastfeeding device of claim 9, wherein the second opening is comprised of a waterproof fastener.

18. A method of using a simulative breastfeeding device, the method comprising the following steps:
providing a simulative breastfeeding device comprised of a garment comprised of an interior pocket that receives at a reservoir comprised of a nipple comprised of an opening;
filling the reservoir with an infant feeding formula or a breastmilk;
opening a vent of the reservoir;
placing the reservoir in the interior pocket;
wearing the garment on a torso of a user; and
breastfeeding an infant via the nipple.

19. The method of using a simulative breastfeeding device of claim 18, wherein the reservoir is comprised of a breast-shaped reservoir.

20. The method of using a simulative breastfeeding device of claim 18, wherein the reservoir is comprised of a silicone area.
